(19) United States
(12) Patent Application Publication
(10) Pub. No.: US 2026/0066082 A1
(43) Pub. Date: Mar. 5, 2026

(54) PREDEFINED SCHEDULING PATTERNS FOR SIMPLIFIED IMPLEMENTATION

(71) Applicant: Boston Scientific Neuromodulation Corporation, Valencia, CA (US)

(72) Inventor: Lisa Denise Moore, Glendale, CA (US)

(73) Assignee: Boston Scientific Neuromodulation Corporation, Valencia, CA (US)

(21) Appl. No.: 19/313,247

(22) Filed: Aug. 28, 2025

Related U.S. Application Data
(60) Provisional application No. 63/689,171, filed on Aug. 30, 2024.

(51) Int. Cl. G16H 20/30; A61N 1/05; A61N 1/36; G16H 40/67

(52) U.S. Cl. CPC G16H 20/30; A61N 1/0529; A61N 1/36139; A61N 1/36167; A61N 1/36192; G16H 40/67

(57) ABSTRACT

Systems and methods for adjusting neural therapy patterns of a neuromodulation system in response to each of patient events and passage of time. Initial stimulation parameters are set and used to start a stimulation period. As time passes or events occur, the neuromodulation system adjusts the stimulation parameters according to a preset temporal pattern. Further examples include handling of patient requests for therapy changes, and acute testing for complex stimulation patterns.

### CROSS REFERENCE TO RELATED APPLICATIONS

The present application claims the benefit of U.S. Provisional Patent Application Ser. No. 63/689,171, filed Aug. 30, 2024, the disclosure of which is incorporated herein by reference.

### BACKGROUND

Neuromodulation therapies have been shown to provide numerous benefits for patients. Various such systems are known, including, for example, systems for spinal cord stimulation (SCS), deep brain stimulation (DBS), Vagus nerve stimulation (VNS), Sacral nerve stimulation (SNS) and/or peripheral nerve stimulation (PNS). Therapy output by such systems is carefully planned and calibrated to a given patient's needs as well as system designs, including implant position, disease state, etc.

Some patients manage their conditions using a combination of neuromodulation therapy and medication therapy. For example, a patient having Parkinson's disease may take medication and also have an implanted DBS system. Throughout the day, the concentration of medication in the patient's bloodstream varies in response to medication consumption, absorption, and the body's metabolization. Likewise, a patient having pain syndrome may take medication and also have an implanted SCS system and, again, the concentration of medication in the patient's system will vary throughout the day. If the DBS system or SCS system provides constant therapy, such patients may be undertreated or overtreated at different times. New and alternative approaches for managing neuromodulation systems to account for changes in therapy need of the patient on a daily basis are desired.

### OVERVIEW

The present inventors have recognized, among other things, that a problem to be solved is the need for new and/or alternative systems and methods for providing complex neuromodulation therapy to a patient.

A first illustrative and non-limiting example takes the form of an implantable medical device system comprising: an implantable medical device (IMD) comprising operational circuitry including a stimulation output circuitry configured to generate therapy outputs and a controller for controlling the stimulation output circuitry; and a lead extending from the IMD and carrying a plurality of electrodes for delivering stimuli to patient tissue, the lead comprising one or more conductors to electrically couple the electrodes to the IMD; wherein the controller is configured to: control the stimulation output circuitry to generate a stimulation pattern; modify the stimulation pattern using a separately defined adjustment parameter for adjusting the applied stimulation pattern; and change the adjustment parameter in response to at least one of: a daily temporal pattern; or a patient event.

Additionally or alternatively, the separately defined adjustment parameter defines an amplitude as a function of time, and controller is configured to modify the stimulation pattern by adjusting an amplitude as a function of time.

Additionally or alternatively, the controller is configured to change the adjustment parameter in response to each of a daily temporal pattern and a patient event by: defining at least first and second time periods, separated by a patient event; prior to the patient event, controlling amplitude of the applied stimulation in response to passage of time according to a first shape; determining the patient event has occurred; and after the patient event, controlling amplitude of the applied stimulation in response to passage of time according to a second shape.

Additionally or alternatively, the system includes a patient device configured to communicate with the IMD, wherein the patient event is a medication being received or consumed by the patient, and the controller determining the patient event has occurred includes the IMD receiving communication from the patient device. Additionally or alternatively, the patient device is a patient remote control configured to be used by the patient to communicate with the IMD and adjust therapy parameters or turn therapy on or off. Additionally or alternatively, the patient device is a drug dispensing device, configured to communicate to the IMD when the medication is dispensed.

Additionally or alternatively, the patient event is onset of sleep or end of sleep. Additionally or alternatively, the IMD is configured to determine onset of sleep or end of sleep using a motion sensor in the IMD. Additionally or alternatively, the IMD is configured to determine onset of sleep or end of sleep by communication with a wearable device on the patient, the wearable device comprising a motion sensor.

Additionally or alternatively, the system also includes a patient remote control configured for receiving inputs from the patient and communicating with the IMD, the system configured for: a) receiving a patient modification from the patient for modifying the stimulation pattern; b) modifying the stimulation pattern using the patient modification to yield a modified and adjusted stimulation parameter; and c) determining whether to retain the patient modification based on comparison of the patient modification to one or more boundary conditions. Additionally or alternatively, the boundary conditions include each of a first boundary and a second boundary, and step c) is performed by: if the modified and adjusted stimulation parameter does not exceed the first boundary, the patient modification is retained and used to modify the applied stimulation pattern until a further patient modification is received, or the implantable pulse generator is reprogrammed; if the modified and adjusted stimulation parameter exceeds the second boundary, the patient modification is used only until one of the following occurs: an inflection point in the daily temporal pattern is reached, and another patient event occurs. Additionally or alternatively, the patient remote control is configured to perform each of steps a), b) and c), and communicate therapy parameters for implementation to the IMD. Additionally or alternatively, the patient remote control is configured to perform step a) and, in response thereto, to communicate the patient modification to the IMD, and the IMD is configured to perform steps b) and c).

Additionally or alternatively, the separately defined adjustment parameter is used to modify at least one of amplitude, pulse width, or frequency of the applied stimulation pattern. Additionally or alternatively, the IMD is a spinal cord stimulator, and the lead is adapted for implantation along the spinal column; or the IMD is a deep brain stimulator, and the lead is adapted for implantation in the brain.

Another illustrative and non-limiting example takes the form of a method of delivering a neuromodulation therapy comprising: applying a stimulation pattern to the patient using an implantable pulse generator attached to an implantable lead, the lead having a distal end carrying a plurality of electrodes, the distal end positioned near a neural tissue to be stimulated; modifying the applied stimulation pattern using a separately defined adjustment parameter for adjusting the applied stimulation pattern; wherein the adjustment parameter changes in response to at least one of: a daily temporal pattern; or a patient event.

Additionally or alternatively, the separately defined adjustment parameter defines an amplitude as a function of time, and the step of modifying the applied stimulation pattern is performed by adjusting an amplitude of the applied stimulation pattern as a function of time.

Additionally or alternatively, the adjustment parameter changes in response to each of a daily temporal pattern and a patient event by: defining at least first and second time periods, separated by a patient event; prior to the patient event, controlling amplitude of the applied stimulation in response to passage of time according to a first shape; determining the patient event has occurred; and after the patient event, controlling amplitude of the applied stimulation in response to passage of time according to a second shape.

Additionally or alternatively, the patient event is a medication being received or consumed by the patient. Such an event may be indicated by the patient using a patient remote control, or may be indicated directly to the IMD or system from a drug dispenser or pump, for example. Additionally or alternatively, determining the patient event has occurred comprises receiving a communication from a patient remote control configured to be used by the patient to communicate with the implantable pulse generator. Additionally or alternatively, the patient event is onset of sleep or end of sleep.

Additionally or alternatively, the implantable pulse generator comprises operational circuitry configured to perform the method, such that the method occurs without communication from a clinician programmer adapted to communicate with the implantable pulse generator.

Additionally or alternatively, the method also includes receiving a patient modification from the patient for modifying the applied stimulation pattern; modifying the applied stimulation pattern using the patient modification to yield a modified and adjusted stimulation parameter; and determining whether to retain the patient modification based on comparison of the patient modification to one or more boundary conditions.

Additionally or alternatively, the step of determining whether to retain the patient modification is performed as follows: the boundary conditions include each of a first boundary and a second boundary; if the modified and adjusted stimulation parameter does not exceed the first boundary, the patient modification is retained and used to modify the applied stimulation pattern until a further patient modification is received, or the implantable pulse generator is reprogrammed; if the modified and adjusted stimulation parameter exceeds the second boundary, the patient modification is used only until one of the following occurs: an inflection point in the daily temporal pattern is reached, and another patient event occurs.

Additionally or alternatively, the separately defined adjustment parameter is used to modify at least one of amplitude, pulse width, or frequency of the applied stimulation pattern.

Further examples include implantable medical devices and/or implantable medical device systems configured to perform the preceding methods.

Another illustrative and non-limiting example takes the form of an implantable medical device system comprising an implantable medical device (IMD) configured to generate therapy outputs, and a patient remote control (RC) configured to communicate with the IMD, the system configured to operate the following method: receiving at the RC a request from the patient to modify a therapy output parameter; analyzing the request and either: determining the request is within a first set of parameter limits and, if so, modifying a programmed setting for the therapy parameter so that the IMD will continue to use the modified programmed setting for the therapy parameter until changed or reset; or determining the request is outside the first set of parameter limits and, if so, modifying the therapy output temporarily and then returning to the programmed setting.

Additionally or alternatively, the system is further configured to modify the therapy output temporarily by modifying the therapy output for a fixed period of time, and then returning to the programmed setting. Additionally or alternatively, the programmed setting varies over time, and the modified programmed setting is mathematically generated to modify a plurality of values of the programmed setting. Additionally or alternatively, the RC is configured to perform the analyzing and determining steps, and issues a communication to the IMD to cause the IMD to modify the programmed setting or to modify the therapy output temporarily. Additionally or alternatively, the RC is configured to communicate the request from the patient to the IMD, and the IMD is configured to analyze the request and perform the determining steps.

Another illustrative and non-limiting example takes the form of a method of testing a therapy program on a patient, comprising: defining the therapy program, including within the therapy program one or more amplitude maximums and one or more amplitude or pattern changes, separated by one or more periods of stimulation; identifying plateau periods as intervals in the therapy program during which none of the one or more amplitude maximums occur and none of the identified amplitude or pattern changes occur, the plateau periods having durations and plateau therapy parameters; creating compressed periods by reducing the reducing the plateau period durations without modifying the plateau therapy parameters; and applying a test program to the patient, the test program being the same as the therapy program except that the plateau periods are replaced by the compressed periods.

Additionally or alternatively, the compressed periods are reduced in duration by at least 75% relative to the plateau periods. Additionally or alternatively, the step of identifying plateau periods comprises: identifying a predetermined number of amplitude maximums, and defining extreme periods for the predetermined number of amplitude maximums, the extreme periods having extreme period durations and extreme period therapy parameters; defining transition periods for the predetermined number of transition period in which the one or more amplitude or pattern changes occur, the transition periods having transition period durations and transition period therapy parameters.

Additionally or alternatively, the method also includes analyzing the transition periods against a slope threshold, and, if the slope threshold exceeds a slope of a first transition period, compressing the first transition period. Additionally or alternatively, the therapy program is a neuromodulation therapy program, and the step of applying the test program comprises either delivering neuromodulation by deep brain stimulation, or by spinal cord stimulation.

Further examples may include implantable medical devices and/or implantable medical device systems configured to perform the preceding methods.

This overview is intended to provide an introduction to the subject matter of the present patent application. It is not intended to provide an exclusive or exhaustive explanation. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows a deep brain stimulation (DBS) system;

FIG. 2 shows an illustrative implantable pulse generator (IPG);

FIG. 3 shows a spinal cord stimulation (SCS) system;

FIGS. 4A-4B show illustrative neuromodulation waveforms;

FIGS. 5-7 graphically illustrate how neuromodulation waveforms can be varied;

FIG. 8 shows a process flow in block form for therapy pattern adjustments;

FIGS. 9A-9D show a user interface for building a temporal pattern;

FIG. 10 illustrates combining parts of a temporal pattern;

FIGS. 11-12 are process flows in block form for determining whether to retain a patient adjustment; and

FIG. 13 is a process flow in block form for initializing and acute testing.

### DETAILED DESCRIPTION

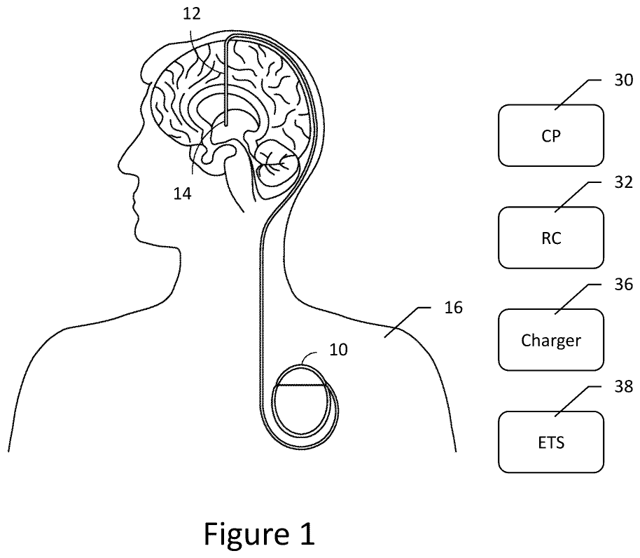

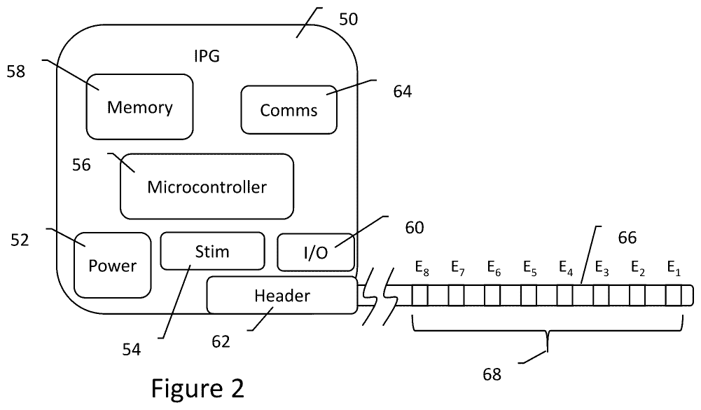

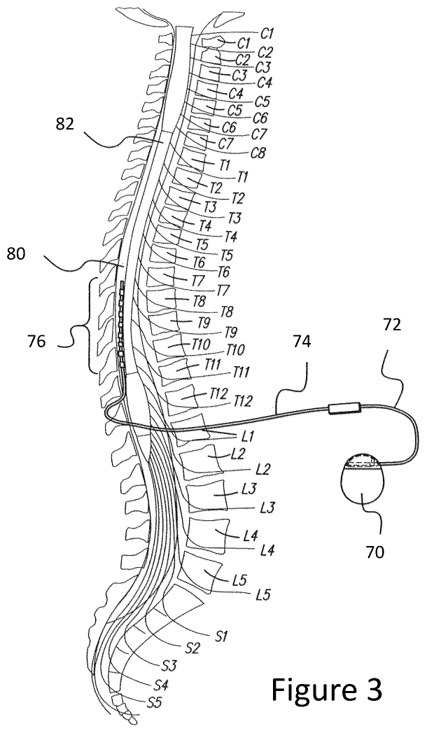

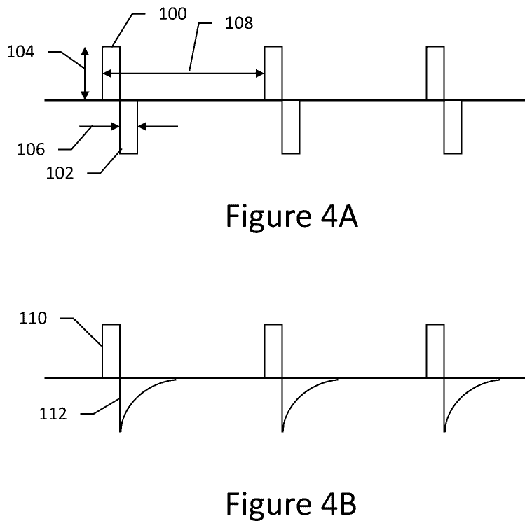

FIG. 1 shows a deep brain stimulation (DBS) system. FIG. 2 shows an illustrative implantable pulse generator (IPG). FIG. 3 shows a spinal cord stimulation (SCS) system. FIGS. 4A-4B show illustrative neuromodulation waveforms. Each of these figures are provided as context for the present invention, and a further described below. These, or other, neuromodulation systems can be used to provide therapy to a patient, and that therapy can be modulated or otherwise controlled using the methods described in greater detail with respect to the remaining Figures.

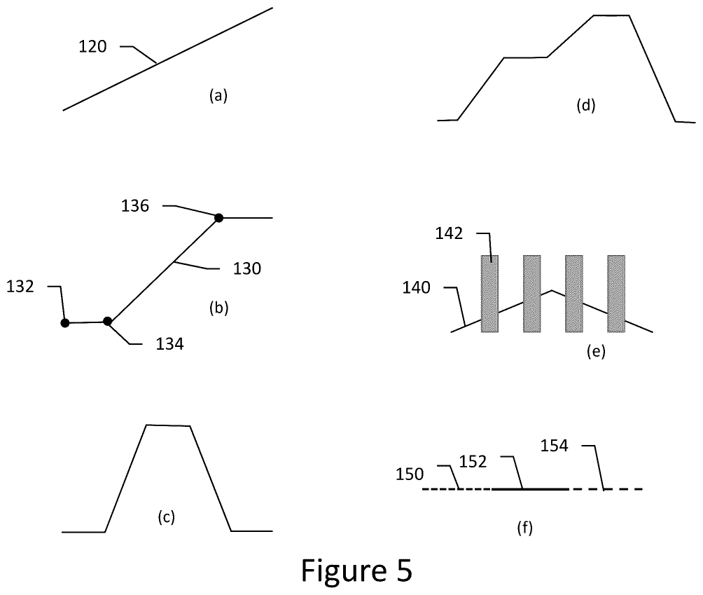

FIG. 5 illustrates several ways in which therapy output by a neuromodulation system can be modulated “up” and “down” in any of current delivered, voltage, current density, amplitude, pulse width, frequency, etc., over time. Therapy delivery may use a setting, such as amplitude, charge density, pulse width, power, and/or frequency, for example, bounded by minimum and maximum settings, and calculated using various inputs. As noted further below, other settings can be used, such as charge per pulse (which would be current amplitude times pulsewidth, for example), or power, average power, etc.

In some examples, a stimulation parameter is varied over time. For example, the amplitude may for stimulus may be determined by:

Min<A*B<Max

Where Min and Max are physician selected (or manufacturer/device imposed) maximum and minimum limits for the amplitude, A is the physician-selected amplitude programmed by the CP, and B is the patient influence programmed by the patient RC. In a defined program for stimulation, a repeatable series of n pulses (p0, p1, . . . pn) may be delivered, wherein the i^{th} individual pulse may have the amplitude:

Min<Aᵢ*B<Max

Where Aᵢ is the i^{th} amplitude as programmed with the CP, and B is again the patient programmed influence. Such a program does not account for time-of-day, and the use of the CP to program an entire day of therapy would be extraordinarily time-consuming with prior art methods, as amplitude would have to be selected using the CP for each pulse and, moreover, the program for such therapy would need to account for a massive number of time instances across a day (for example, using a 256 Hz sample rate, the program would set amplitudes for 256 Hz*60 sec/min*60 min/hr*24 hr/day, or over twenty-two million samples. While programming may use other approaches, the need to program 24 hours of therapy is not desirable.

Instead, the present disclosure is directed to adding to the program a separate variable. For simple tonic stimulation, accounting for patient input (which may be optional), the amplitude or other controlled parameter at any given point in time may be the product shown here:

Min<A*B*C(t)<Max

Where, again, Min and Max are the upper and lower limits programmed by the CP, A is the programmed parameter from the CP, B is the patient influence from the RC, and C(t) is a time varying function. The time varying function C(t) may be described as a separately defined adjustment parameter or variable, and is used to modify an underlying therapy program over time. For a more complex program setting, the amplitude or other controlled parameter may be as shown here:

Applied Parameter=Min, if Aᵢ*B*C(t)<Min Aᵢ*B*C(t) Max if Aᵢ*B*C(t)>Max

Here, a mixed function is shown to simplify the formula. In implementation, the time varying function C(t) may be converted to a discrete value using any suitable conversion, as is known in the art for discretizing a continuous function. In some examples, discretization may be performed by the CP and downloaded to the IPG, for example. Discretization may occur in the IPG if desired.

The t in C(t) may refer to a clock time, or may refer to the time since a predefined event was recorded, such as, for example and without limitation, an indication that the patient begun an activity, or an indication that the patient has consumed a medication. For example, C(t) may use clock time and reset every 24 hours in some examples. In some examples, C(t) may use events, for example, wakeup, morning medication, afternoon medication, and sleep time. If the C variable is event-based, the discretization may store several individual time series to correspond to each of several segments.

An indication that the patient has begun an activity may be determined by, for example, using an accelerometer in the IPG to sense patient movement (waking, initiation of exercise), lack of patient movement (sleep onset), or by using the RC if, for example, the RC is embodied as an application operating on the patient's smartphone which may, for example, determine a patient activity or medication consumption using an input from the patient received by the application, determine waking of the patient if the patient turns of an alarm or starts using the device (i.e. Internet browsing, texting, playing a game or social media activity). In a connected device scenario, for example, the patient may receive medication from connected dispenser, such as a Bluetooth equipped drug pump or patient medication holder, and in either case the RC (or even the IPG) may communicate with the drug pump or the medication holder to determine the medication has been dispensed. In another connected device example, the patient may wear an activity monitoring device, such as a watch, having an accelerometer that can detect movement, or the lack thereof, to determine sleep or waking state

Rather than a function or calculation-based approach in which a parameter of therapy is modified, in some examples, the stimulation pattern or program may change. For example, with some systems, there may be desire to provide a first stimulation pattern while the patient is awake to alleviate symptoms, and a second stimulation pattern while the patient is asleep to reduce or arrest disease progress. An example may include programmed patterns for symptom alleviation and anti-neural-inflammation effects, as suggested, for example, in US Prov. Pat. App. 63/543,193, filed Oct. 9, 2023 and titled SYSTEMS AND METHODS FOR MODULATING THE NEUROIMMUNE SYSTEM, the disclosure of which is incorporated herein by reference. More broadly speaking, a first stimulation pattern may be used to achieve a first goal, and a second stimulation pattern may be used to achieve a second goal, and the system may be configured to switch from the first stimulation pattern to the second stimulation pattern, if desired. Thus, at a desired point in time, or in response to a defined event, the present methods/systems may be used to switch from one program/pattern to another in some examples.

While amplitude and pulse width can be independently controlled parameters, other “compound” parameters may combine amplitude, pulse width and/or repetition rate or frequency together as a compound parameter. For example, charge per pulse may be a controlled compound parameter, determined as the product of amplitude and pulse width. Average current magnitude can be used as well, in which case the magnitude of current per unit time (or per pulse period) can be controlled. In some examples, to limit accommodation and/or to encourage patient response, one or more of amplitude, pulse width and/or frequency may vary over time while maintaining a constant (or near-constant, within preset bounds) average power, voltage or current, as desired. In a system using such a waveform, the controlled parameter may be a compound parameter such as any of current per pulse, the product of voltage and pulse width, average power, average voltage, or average current. Root-mean-squared current, voltage or power may be used if, for example, a continuous waveform is delivered.

FIG. 5 shows several examples of a temporal progression through a series of pattern changes. At 5(a), the pattern 120 simply increases the controlled parameter, such as pulse width or amplitude, as a function of time. At 5(b), the applied pattern defines inflection points or corner points (either can be used) at 132, 134 and 136, triggering changes in the controlled parameter as line 130 is followed. For example, 132 may be an inflection point in the temporal pattern corresponding to a pre-wakeup period, which may be clock triggered. At 134, the inflection point can correspond to detected patient wakeup, such as by detecting patient movement, receiving a patient input, or monitoring the patients use of a connected device such as a smartphone. Therapy then ramps toward a levelling off point at 136, which may be clock derived or may correspond to the patient receiving medication or other patient activity or input. Other shapes may have a plateau as shown at 5(c), with later drop-off due to patient medication consumption or sleep onset. Multiple tiers can be used as shown at 5(d), where initial ramping may be to allow the patient to adjust in the morning, with a flattened portion after medication has been taken, and subsequent increase in the afternoon, and dropping off at bed time. At 5(c), off periods 142 are shown interspersed with the waveform 140. Off periods 142 may be clock triggered or event triggered, as desired.

Finally, at 5(f) the temporal pattern may go through several different therapy/pattern types over time, with different therapy applied at 150, 152, 154. Therapy changes in FIG. 5(f) may, for example, use different electrode combinations, different frequencies, amplitudes or pulsewidths, as desired. One pattern may be a burst pattern, having sets of therapy pulses with relatively shorter inter-pulse periods, separated by relatively longer inter-burst periods. The shapes shown in FIGS. 5(a) to 5(f) may represent the separately defined parameter as used elsewhere herein, for example, C(t) as described previously.

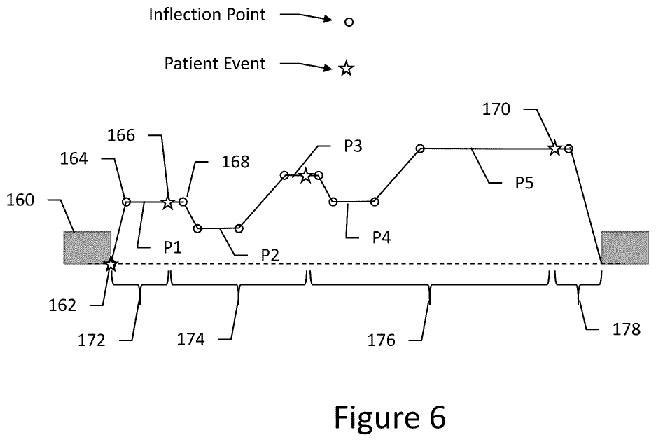

FIG. 6 shows a temporal pattern with several steps and changes therein. As used herein, an inflection point in a therapy pattern is a time at which the therapy pattern changes. This may not necessarily be a change in the therapy, but is instead a change in the therapy pattern, as illustrated in FIG. 6. The inflection point may refer to the point in time in which the therapy pattern changes as the separately defined parameter, such as C(t) used above, changes, or when the slope of C(t) changes.

Starting from the left, therapy is off as shown in the grey box at 160. A patient event occurs at 162, which is simultaneously an inflection point in the therapy (the circles are used for inflection points; the circle at 162 is obscured by the star 162 indicating the patient event). For example, at 162, the patient may be observed as having woken up through one of several means. Patient waking may be detectable by changes in patient heart rate or posture, onset of activity, or the patient initiating use of a smartphone, for example and without limitation. For example, the implantable device may be adapted by detect heart rate, or may be in communication with a device, such as a smartwatch or other communication-enabled implantable or wearable device; when heart rate changes (possibly combined with consideration of time of day, as disclosed in commonly assigned U.S. Prov. Pat. App. No. 63/688,511, titled CREATION AND MAINTENANCE OF TIME AND TRIGGER REACTIVE SCHEDULES, filed on Aug. 29, 2024, the disclosure of which is incorporated herein by reference), the implantable device may use the change in heart rate to indicate patient wake-up. In other examples, an implantable device may have an accelerometer that can be used to determine patient posture and/or activity indicating transition from sleep to waking. As noted, the patient's use of a device such as a smartphone may be treated as a patient event; for example, some systems use the patient's smartphone as a patient remote control, taking advantage of ubiquitous Bluetooth technology that can also be provided in the implant. In other examples, the patient event at 162 may be activation of therapy in response to expiration of a timer, noting, for example, a patient may begin receiving therapy prior to waking if desired, to anticipate and alleviate symptoms. In other example, therapy can be initiated using a patient remote control, which may or may not be the patient's smartphone.

From the patient event and inflection point at 162, the therapy parameter increases until a period of time expires, or a parameter target is met, as indicated at the inflection point at 164. Inflection point 164 stops the upward ramp of the parameter, which remains flat. The controlled parameter may be, for example and without limitation, any of various selectable parameters, including therapy amplitude, pulse width, frequency (pulses per second, for example), or other suitable parameter. In some examples, power or energy per unit time can be controlled, with the waveform itself varying in other parameters (as with a stochastic generator, noise-type signal, etc.). The examples that follow will focus on therapy amplitude, but it should be noted that these other parameters may be used in other examples, and control over amplitude is not required.

A patient event occurs at 166. Here, the patient may, for example, use a smartphone or patient RC to communicate to the system that she has taken medication. Medication may reduce the need for the therapy from the implantable device, but also takes time to be absorbed into the body and take effect. Thus, a time period is defined from patient event 166 to the subsequent inflection point at 168. At 168, the pattern of therapy changes, and the controlled parameter begins to drop until reaching a lower value, as shown graphically. Further inflection points may be based, for example, on time since a prior inflection point, or time since a patient event, for example. A patient event also takes place at 170. This patient event 170 may be, for example, an indication that the patient has gone to bed, and in response the therapy parameter ramps downward until therapy turns off, as indicated again by the grey block.

The therapy pattern is simplified by a building-block approach based in part on FIG. 5. Segments are defined at 172, 174, 176 and 178, each triggered by a patient event. The segments 172, 178 in this example each have a shape as defined at FIG. 5(b)—that is, a first plateau having a first parameter value, a ramp, and a second plateau having a second parameter value that is different from the first. Each of the first plateau, and ramp have a defined duration, while the second plateau may have a defined duration or may be configured to start at the end of the ramp, and end with a patient event. Segments 174 and 176 are more complex, and resemble the pattern of FIG. 5(e) instead, with three ramps and three plateaus.

Treating the controlled parameter as therapy amplitude, segment 172 has a first plateau with zero duration, defining a ramp from zero amplitude to a first target amplitude, the ramp having a duration of, for example, 15 minutes, as this is the wakeup-onset in an example. The second plateau is at the first target amplitude, and has an indeterminate duration, ending at the patient event 166. The second segment 172 has a first plateau at the first target amplitude, with a duration as shown from 166 to 168, for example, ten minutes, after which the ramp starts at the first target amplitude and goes down to a lower second target amplitude, with a ramp duration of, for example, five minutes. The therapy amplitude remains at the lower level for a period of time, for example, two hours, before ramping upward again to a third target amplitude.

The approach shown in FIG. 6 is to break up the therapy pattern throughout the course of the day, allowing a modular programming method to be used. One or more of the patient events may be replaced with a simple timing-based event; for example, the wakeup 162 may be at a set time of day, if desired. By introducing the modular programming method, a therapy parameter can be controlled throughout the day without adding an arduous programming task for the physician or user.

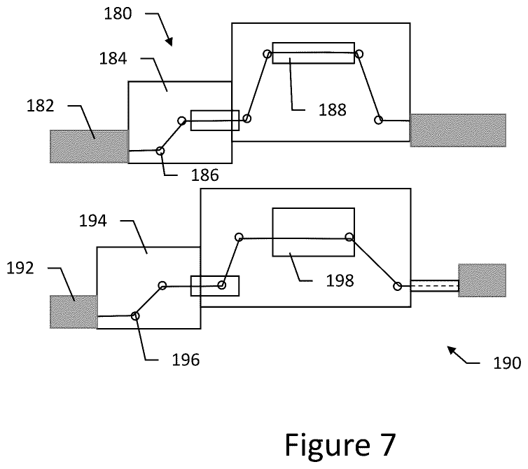

FIG. 7 shows a temporal pattern across two channels. A first channel 180, such as a first DBS lead, may use a pattern as shown. Again, grey boxes 182, 192 indicate therapy off. One or more time windows 184, 194, which may be driven by patient events or may be based on time of day, are defined in each of the channels, creating segments in which user-selected patterns are applied. Inflection points 186, 196 indicate the points in time in which the therapy pattern changes.

One aspect of the illustration in FIG. 7 is that each of the two channels 180, 190, operates independently of the other. Thus, starting points, ramps, plateaus, etc. in each channel do not rely on the action in the other channel. Moreover, any modifications made in one channel can be performed without affecting the other channel, and patient events or inflection points in each of the two channels can also be independently defined.

A further concept provided in FIG. 7 is that of the micro and macro modifications that can be defined. It is known in the art to allow the patient, using a patient remote control, to request and obtain changes to therapy parameters, most often, therapy amplitude, on their own but within limits defined by the physician. Macro ranges refer to outer boundaries of therapy defined by the physician (these may align with but are not the same as device operational limits), while micro ranges refer to a narrower set of boundaries. The patient is allowed, in some examples, to modify the controlled therapy parameter within macro ranges and no further, based on physician definitions for the macro ranges. If a patient modification is within an associated micro range, the patient's change will be retained. The change may be retained in several ways, as further discussed below.

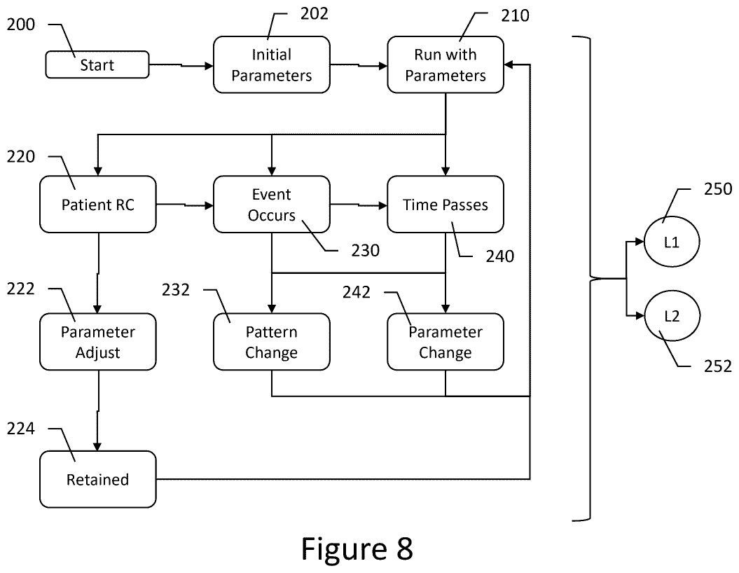

FIG. 8 shows a process flow in block form for therapy pattern adjustments. Start block 200 may be triggered, for example, by the patient initiating or activating a therapy program. Initial parameters 202 are then loaded and the system begins to operate or “run” 210 using the current parameters, which would be those loaded at 202 for the first iteration, but then change in further iterations.

As the system operates using a set of parameters, those parameters can be modified through one of several methods. A first way the parameters can change is via the patient remote control (RC), as indicated at 220. As described previously, the patient may be allowed to modify one or more parameters of therapy, as indicated at 222. Those parameter changes implemented by the patient are analyzed to determine whether the parameter adjustments are to be retained, as indicated at 224. With therapy parameters modified, at least temporarily, the method returns to block 210 and continues to deliver therapy.

In operation, for example, the programming may be configured so that the parameter adjustment at 222 is paired with stored data indicating an end point, end time, or other basis for termination of the parameter adjustment, such as by indicating that the parameter adjustment is to terminate when some event occurs (230), or time passes (240), or another patient RC interaction (22) with the system. On the other hand, if the parameter adjustment is to be retained, the parameter adjustment at 222 is not paired with the stored data, and is instead acted upon by adjusting the stored program. In still other examples, the parameter adjustment may be stored in one or more registers of data that are marked with different variable types so that the parameter adjustment can be marked as retained or not retained going forward. The skilled person will recognize several ways that such retained, or not retained, parameter adjustments can be implemented.

The process flow returns to 210 unless interrupted, for example, by the patient RC stopping the program, or if some fault or other interaction takes place, such as interrogation with the clinician programmer (CP), or, for rechargeable systems, if the device is subjected to a charging operation, in which case therapy may be suspended (though this need not be the case and is merely an example).

The run state at 210 iterates upon itself, executing the stored program, such as a tonic, burst or other neuromodulation therapy. If a patient event occurs, as indicated at 230, the process flows through block 230 to either block 232, in which a pattern change takes place, or block 242, in which a parameter change is executed. The patient event may include, for example and without limitation, detection or indication of patient wake-up, such as by the implanted device detecting wakeup/movement, or by the patient indicating wakeup through the patient RC 220 (thus the line from 220 to 230). Other patient events may include the patient taking or receiving a medication, where the patient may provide an indication via patient RC 220, or where a medication dispensing device (an implantable device, or a pill dispenser having Bluetooth capability for example). A patient event may include onset of patient activity, such as exercise. In another example, a patient event may include the patient directly interacting with the implant, such as by tapping the device though the skin, an action that may be detected by an accelerometer in the implanted device; tapping with a selected pattern or determined number of taps may be used as a way of the patient indicating, for example, a desire for therapy to start, or that medication has been taken, if desired. Other examples of a patient event have been given above and below in this disclosure, and may be used at 230.

A pattern change 232 may include, for example, a change in the stimulation pattern that switches from one pattern of therapy to another. For example, tonic therapy may be substituted for burst therapy, or the other way around, representing a pattern change 232. A step in the pattern shown at 5(f) above, is an example, as one therapy design is substituted for another. A pattern change 232 may include re-weighting or replacing of the electrodes used for therapy. Steering a central point of stimulation (that is, the mathematical centroid of either the cathodic or anodic current, for example) to a new location may be a pattern change in some examples.

A parameter change 242 may instead occur in response to an event occurring at 230. A parameter change may be any of the above described changes or adjustments, such as changing therapy amplitude, frequency/repetition rate, pulse width, or compound factors such as charge density, energy or power per unit time, etc. A difference here is that the change at 232 and 242 are not treated the same as a parameter adjustment 222 from the patient RC, as there is no need for analysis to determine retention.

Again, the process will return to block 210. Another way to reach a pattern change 232 or parameter change 242 is that time passes, as indicated at 240. Time passing 240 may take several forms. Time may be measured against a 24-hour clock, for example, looking at specific time points throughout the date. Time can instead be measured as time from a prior event, such as time from an event occurring 230. An example is shown above in FIG. 6, as a patient event occurs at 166, and a delay is imposed; once sufficient time has passed after event 166, the therapy changes as indicated at 168, with the inflection point at 168 representing passage through block 240 in FIG. 8 to either of blocks 232, or 242.

As highlighted n FIG. 7, above, the process flow of FIG. 8 may be operated more than once in parallel. For example, the process flow may be performed for each of a first lead 250 and a second lead 252, such as in a DBS system with bilateral lead positions. Rather than leads L1, L2, as shown, different areas or targets may be represented. For example, a system can be implanted in a patient having each of a spinal lead in the lumbar region and another lead in the thoracic region. In still other examples, different areas may be defined as 250/252, such as using two different subsets of the electrodes on a paddle lead implanted near the spinal column. Any configuration with separately programmable areas or subsets of electrodes may be represented at 250, 252.

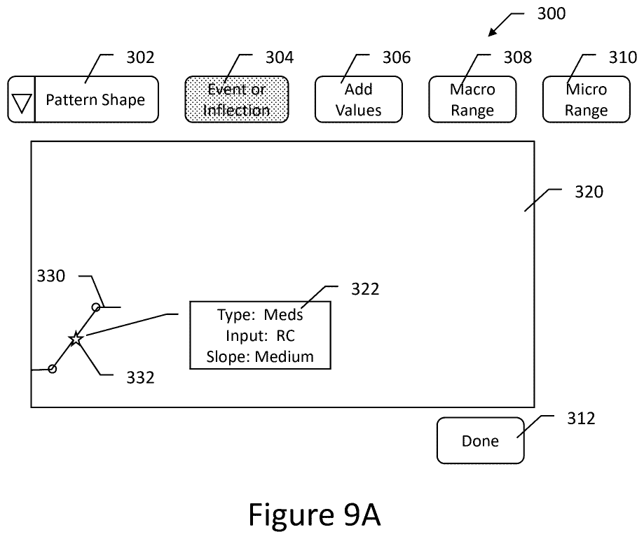

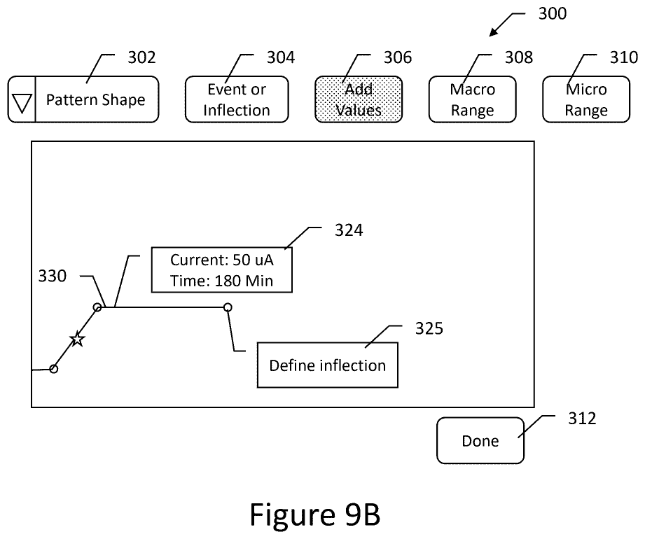

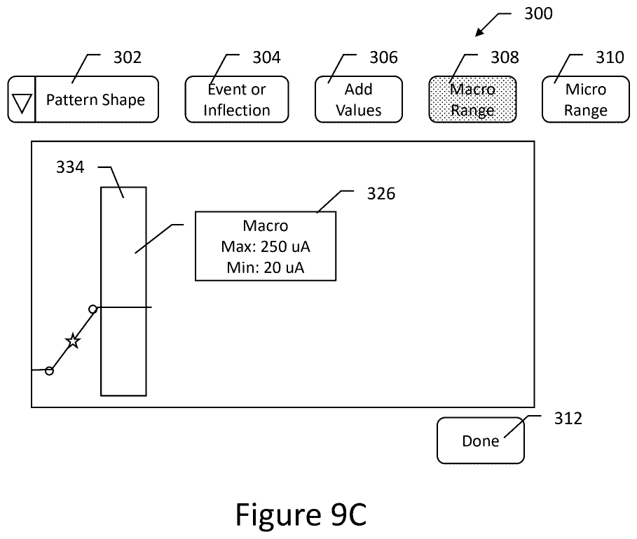

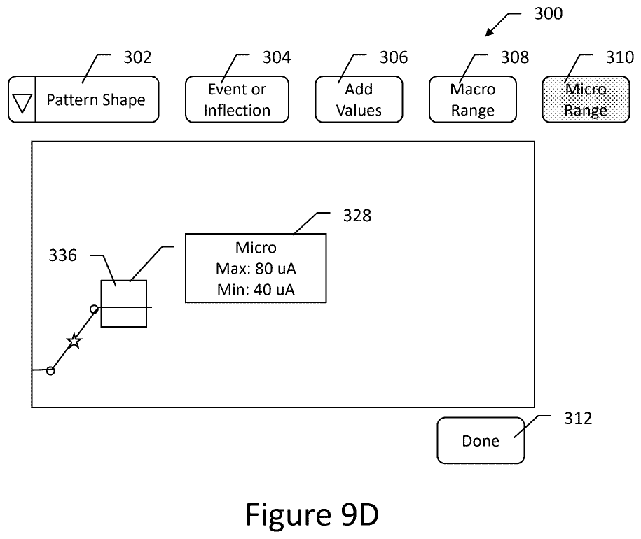

FIGS. 9A-9D show a user interface for building a temporal pattern. The user interface 300 may be implemented on a touchscreen for a clinician programmer, and is designed to simplify the approach to programming complex changes throughout the day and/or in response to patient events. A pattern shape icon button is shown at 302, and may take the form of a drag-and-drop menu or list of shapes. The pattern shapes may be as shown above in FIG. 5, with options specific to each shape and/or points in the shapes. As shown in FIG. 9A, a shape 330 has been selected and dropped to the far left or beginning point in time. The next icon, 304, allows the user to select to drag and drop either an event indicator or an inflection point marker. Icon 304, when selected, allows the user to place and drag into a desired position an inflection point or patient event. Further definitional information can then be provided by the user/physician once the event or inflection point is positioned as desired. For example, as shown at 322, the user, once the event is positioned where needed, is allowed to provide a description, source of input data, and slope information (low, medium, high) in this example. In other examples, the slope may be defined by surrounding plateaus or other data points, if desired. Here, it may be noted that the patient event can occur even on a slope, rather than on a plateau; if the event is not detected, the user may be enabled to also define, for the slope, a conditional endpoint or peak value, in the event the event is not detected or received.

Turning to FIG. 9B, icon 306 may be activated by the user to allow values to be added as desired throughout the waveform. Here, the user has selected icon 306, and then tapped on the flag region shown, and box 324 opens up. In the example, the user is allowed to define a current value and duration for the segment that is selected. Because the user has selected a specific duration, something will happen at the end of that duration, so another pop-up will appear to ensure the user next defines what happens next, as shown at 325. If therapy termination is desired at 325, the user can so indicate by first tapping on box 325, and then selecting the “Done” icon 312, for example.

In FIG. 9C, the user has now selected the Macro Range icon 308, which allows the user to drag/slide the edges of box 334, or populate data in block 326 manually, to define the macro range associated with a chosen segment, as shown. A macro range 334 may be used to define the maximum patient modifications that will be accepted for a given parameter, such as amplitude, pulse width, frequency, current density, and/or power or energy per unit time. Alternatively, a macro range may define the maximum patient modifications that will be retained, rather than used only once. In FIG. 9C, on the other hand, the user has selected the icon 310 for the micro range. Again, dragging to resize box 336 may be used to select the micro range, or values may be entered manually as shown at 328. The micro range may indicate limits applicable to patient modifications that determine whether the patient modification will be retained, as discussed herein, or discarded. The preceding and following discussions of retaining patient modifications apply to this process.

In some examples, micro and macro ranges can be paired in different ways:
In one example, a micro range is a range in which a stochastic or other random or semi-random variation of the applied therapy signal is allowed, while the macro range is a range in which patient modifications will be accepted and retained, wherein patient modifications exceeding the macro range are used only once.
In another example, a micro range is a range in which all patient modifications to therapy parameters are retained, and a macro range defines maximum patient modifications that will be accepted, wherein patient modifications outside the micro range but inside the macro range are implemented once, for a fixed period or for a period that ends with a patient event.
   Other ways of defining and using two ranges in a system adapted for patient modification may be used.

The user interface of FIGS. 9A-9D may be understood as a pattern builder user interface. The pattern builder user interface may be displayed on a screen or touchscreen associated with user controls, such as a keyboard, mouse, trackpad, roller ball, etc. Block 302 is a drop-down pattern shape icon allowing the user to select from various shapes, including flat, ramped (up or down), or more complex patterns, including curved patterns. Event or inflection points can be placed using icon 304; that is, once icon 304 is activated, the user then touches or clicks at a chosen location along a displayed pattern shape and may be queried as to whether a patient event or an inflection point is desired at the location. Options for the patient event type are then presented, if patient event is chosen, and options, including triggers and/or delays for the inflection point are presented, if inflection point is chosen. Specific values may be added for any particular point, whether an inflection point, end point, plateau, etc. by selecting icon 306. When icon 306, and/or icons 308 and 310 are selected, the type of parameter to be controlled can also be selected from a list, including pulse repetition rate, pulse width, pulse amplitude, for example. If desired, the add values tab 306 may also allow the user to choose among waveform shapes if, for example, active recovery pulses (FIG. 4A) are delivered in one time period, and passive recovery periods (FIG. 4B) in another time period (points switching from one waveform shape to another would be inflection points). Swapping among pulse types may include switching between discrete pulses (square waves, sawtooth or triangle waves, etc.) and continuous waveforms (sinusoidal for example); again, icon 306 can be used for such pulse type switching in an example, though other examples may have additional icons, or icon 306 may be a drop-down menu with different value/or type characteristics selectable. Icons 308 and 310, when selected, allow a user to identify a point or plateau on the displayed waveform, and set, such as by dragging and dropping, or by entering specific values, any desired micro- or macro-boundaries.

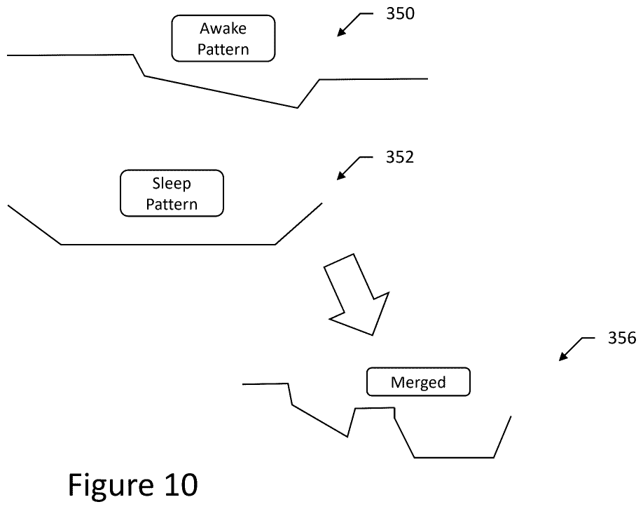

FIG. 10 illustrates combining parts of a temporal pattern. The builder shown in FIGS. 9A-9C can be used to generate one or more segments of therapy programs. Scrolling left to right may be used, if desired, to define an entire 24 hour cycle, which can then repeat. Alternatively, subparts can be defined first, and then concatenated or otherwise merged. In FIG. 10, an awake pattern is defined as shown at 350, as is a sleep pattern at 352. These can then be merged, as shown at 356.

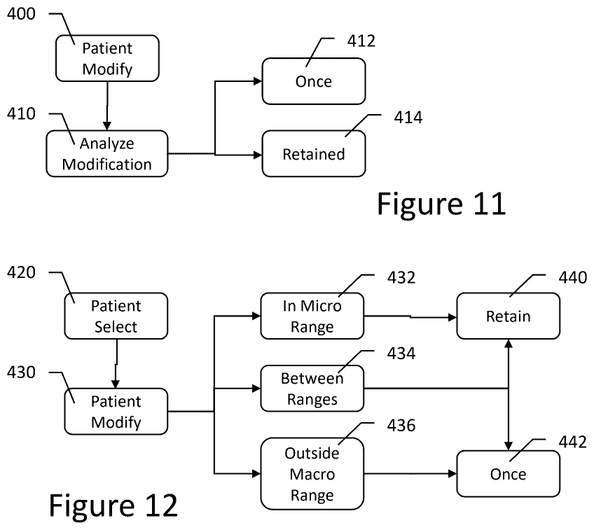

FIGS. 11-12 are process flows in block form for determining whether to retain a patient adjustment. In FIG. 11, the patient selects a parameter to adjust at 400, and the adjustment is then analyzed at 410. Depending on the details of the adjustment, the adjustment may be used only once, as shown at 412, for example for a set period of time (minutes to hours) or until a next inflection point or patient event in the therapy regimen. The adjustment may instead be retained at 414, and reapplied/reused in subsequent iterations (days) of the therapy regimen.

FIG. 12 provides a more detailed approach. Here, the patient selects a parameter at 420 and requests a modification, as indicated at 430. If the modification is within a predefined micro range, at 432, the modification will be retained for further use. If the modification is outside the predefined macro range, as indicated at 436, the modification is used only once. Modifications lying between the micro and macro ranges (that is, inside the macro range, but outside the micro range) pass through block 434, can be retained 440 or used only once 442, based on user choice and/or based on a learning system. For example, a modification falling in block 434 may occur on a given day. The first time the modification occurs at the patient request, it would be used just once. If the modification is repeated, however, for example, 3 times within a period of 5 days (or other X/Y approach), or more than once in a given day, the modification would then be retained once some repetition threshold is reached. The modification request may be “similar” and not necessarily identical, using predefined criteria to determine what modifications would be considered similar.

When a patient modification to the therapy pattern is retained, this may be used in several ways. For example, the patient modification may be applied only to the plateau within which the patient made the change. Thus, referring to FIG. 6, the patient may modify the therapy parameter during the interval between 164 and 168. If the modification is retained the retained modification may only apply that that one plateau within the overall therapy program. The modification may apply to each of the plateau and the endpoints of the ramps on either side thereof. Each time the therapy program restarts, this modification will be retained, so that if the patient makes the change on a Monday, that retained modification will be implemented again on Tuesday, Wednesday and so forth.

In some examples, outside of the one segment that the retained modification occurred within, the therapy pattern may not change. Alternatively, a learning algorithm can be applied. Using FIG. 6 still as the example, supposing the patient makes a retained modification in the plateau P1, between 164 and 168, in a learning example, the retained modification is applied to each subsequent part of the overall pattern, that is, to plateaus P2, P3, P4 and P5. Because each ramp segment between plateaus has start and end points defined by the plateaus, the ramp segments likewise would be modified. However, the learning part is that if the patient makes a later change, for example, during plateau P3, the retained modification from P1 would only apply to P1 and P2 and the ramp therebetween. An endpoint of the ramp leading to P1, and the ramp coming from P2, would also be modified. If the modification in P3 fits in the micro-range for P3, then that modification would also be retained, and would apply to P4 and P5, unless the patient again intervenes. If the modification in P3 does not fit in the micro-range for P3, then that change is not retained. Because the modification occurs in P3, however, the retained modification made during P1 would be applied only to P1 and P2 and would not affect P3. The “learning” part is that the system would learn from the patient interaction how long to retain each modification within the therapy program.

At the implementation level, FIG. 12 can be executed in more than one way in an implantable medical device (IMD) system. The patient selection and modification at 420 and 430 can be performed using the patient remote control (RC), which may be a dedicated device or can be a multi-purpose device operating an application for the IMD system, such as a smartphone. That is, the patient selects 420 a program or parameter to modify, and makes the request for modification 430 to the selected program or parameter. The RC may then perform the analysis of the modification, using buckets at 432, 434, 436 to determine next steps. If the modification is to be retained 440, the RC may communicate this to the IMD by, for example, commanding the IMD to modify a stored therapy program. For example, if simple tonic stimulation is in use and the change is an amplitude or pulse width which is constant in the stored therapy program, one value is updated—that is, the amplitude the whole program or pulse width, for example. If a more complex program is in use, such as one which varies amplitude over time, a plurality of stored values in the program can be modified, such as by using a percentage change in amplitude or other parameter applicable across multiple stored values. The skilled person will recognize several ways such a process can be implemented. Moreover, a program can include, for example, multiple values that are used in a sum or product to determine a parameter, including amplitude, using the products or sums as illustrated above. If a temporary modification is made, instead, the temporary change can be implemented for a period of time in the IMD, with the command from the RC causing the IMD to modifying the therapy parameter, but to retain the therapy program, for example, allowing subsequent return once a time limit for the modification has expired. The time limit may be, for example, a preset period in terms of minutes to hours, if desired. In some examples, when a program is in use, the temporary change remains in place until the program reaches an endpoint, such as keeping a temporary modification in use until the patient goes to sleep at night, or takes a medication, or reaches an inflection point in the therapy regimen as described herein. In other examples, the RC can be used by the patient to perform the steps at 420, 430, and the subsequent analysis and therapy modification is performed in the IMD. If a modification is retained, the IMD may preserve both the modification as well as data indicating a previous state of the programmed parameters, allowing reversion at the option of the patient and/or physician.

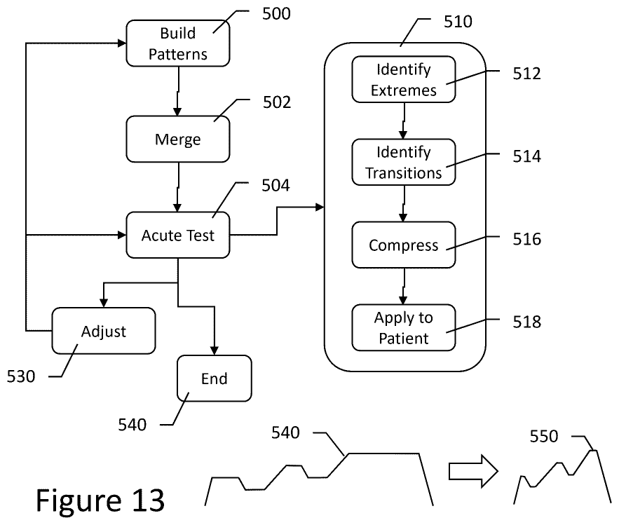

FIG. 13 is a process flow in block form for initializing and acute testing. In the process, patterns are built by a user at 500, such as using the builder shown in FIGS. 9A-9D, or by any other suitable method. These patterns may then be merged 502, such as shown in FIG. 10. Optionally, block 502 can be omitted if desired and block 500 includes building one schedule which can be operated daily or for some lesser period of time, for example, for just the patient's waking hours or sleeping hours, or for a quantity of hours (4-12 hours, for example).

Acute testing of the resulting therapy regimen is then performed as indicated at 504. If there are adverse effects to the patient in the acute testing, adjustments are made at 520. Adverse effects here may include, for example, onset of dizziness or tremor, or other undesired outcome. The acute test 504 is not necessarily intended to prove the therapy regimen works, but is instead used to ensure that therapy parameter changes are accepted by the user without undue difficulty. For example, steep ramps or changes in therapy parameters may cause adverse effects to the patient.

The acute testing 504 can be performed on a condensed/compressed waveform. The acute testing 504 focuses on extremes of the therapy parameters, and changes to therapy parameters, and how these aspects of the therapy pattern affect the patient. An example is shown at 510. First, extremes of the therapy delivery are identified at 512. The extremes are the high and low values of therapy as delivered. Macro/micro ranges need not be tested, as those are used in response to patient input, at least in some examples. Other examples may test at least the micro ranges and/or macro ranges.

Transitions are identified at 514. Transitions include ramps up and down throughout the various steps of the therapy. The overall waveform is compressed. Most of the compression is applied by reducing the duration of static parts of the therapy regimen—that is, durations identified as plateaus above. The compression 516 may, for example, turn a 24 hour therapy regimen into an acute test that can be executed in under one hour, preferably while the patient is being observed in-clinic or in-hospital. The compressed regimen is then applied to the patient, as indicated at 518.

To illustrate the acute testing, FIG. 6 is again helpful. Plateau periods are identified at P1 to P5 in the annotated waveform. Such periods are identified, in an illustrative example, by the use of one or more constant therapy parameters in time periods having either extremes or transitions at either end. The extremes are preserved, as are the transitions, in an example. If desired, transitions may be adjusted to increase slope, such as enforcing a minimum slope requirement. On the other hand, amplitude of output waveforms, or waveform peak amplitudes, may be constant across a plateau, so it may not be as helpful when trying to identify an adverse reaction in an acute test 504 to allow such plateaus to run. By identifying periods of time in which the amplitude is generally unchanging, the plateaus P1 to P5 can be defined, and then compressed in block 516. This means, for example, that an overall pattern of amplitude as shown at 540 would be compressed to the pattern shown at 550, thus reducing the 24-hour (if full day), or 12 to 16 hour (if waking hours) duration of the daily pattern to something which can be applied during an office visit, such as in the range of four hours or less, more preferably, two or even one hour or less. At least a 50% reduction in total time, and a 75% reduction in plateau periods may be applied, in some examples.

Returning to the first figures, FIG. 1 shows an illustrative DBS system implanted in a patient. The system comprises an implantable pulse generator (IPG) 10, shown implanted in the pectoral region of a patient 16. The IPG 10 is coupled to a lead 12 which extends subcutaneously to the head of the patient 16, through a burr hole formed in the patient's skull, and then into the brain. In the example shown, the lead 12 includes a plurality of electrodes positioned near the distal end 14 of the lead. The lead 12 may be placed at any suitable location of the brain where a target for therapy is identified. For example, a lead 12 may be positioned so that the distal end 14 is near the mid-brain and/or various structures therein that are known in the art for use in providing stimulation to treat various diseases.

DBS may be targeted, for example, and without limitation, at neuronal tissue in the thalamus, the globus pallidus, the subthalamic nucleus, the pedunculopontine nucleus, substantia nigra pars reticulate, the cortex, the globus pallidus externus, the medial forebrain bundle, the periaquaductal gray, the periventricular gray, the habenula, the subgenual cingulate, the ventral intermediate nucleus, the anterior nucleus, other nuclei of the thalamus, the zona incerta, the ventral capsule, the ventral striatum, the nucleus accumbens, and/or white matter tracts connecting these and other structures. Data related to DBS may include the identification of neural tissue regions determined analytically to relate to side effects or benefits observed in practice. “Targets” for DBS may include brain structures associated with therapeutic benefits, in contrast to avoidance regions or “Avoid” regions which are brain structures associated with side effects.

Conditions to be treated may include dementia, Alzheimer's disease, Parkinson's disease, dyskinesias, tremors, depression, anxiety or other mood disorders, sleep related conditions, seizures, Epilepsy, etc. Therapeutic benefits may include, for example, and without limitation, improved cognition, alertness, and/or memory, enhanced mood or sleep, elimination, avoidance or reduction of pain or tremor, reduction in motor impairments, seizure management, and/or preservation of existing function and/or cellular structures, such as preventing loss of tissue and/or cell death. Therapeutic benefits may be monitored using, for example, patient surveys, performance tests, and/or physical monitoring such as monitoring gait, tremor, seizure, etc. Side effects can include a wide range of issues such as, for example, and without limitation, reduced cognition, neuroinflammation, alertness, and/or memory, degraded sleep, depression, anxiety, unexplained weight gain/loss, tinnitus, pain, tremor, etc. These are just examples, and the discussion of ailments, benefits and side effects is merely illustrative and not exhaustive.

The illustrative system of claim 1 includes various external devices. A clinician programmer (CP) 30 may be used to determine/select therapy programs, including steering (further explained below) as well as stimulation parameters. The CP 30 can be used by a physician, or at the direction of a physician, to obtain data from and provide instructions the IPG 10 via suitable communications protocols such as Bluetooth or MedRadio or other wireless communications standards, and/or via other modalities such as inductive telemetry. Stimulation parameters may include amplitude of stimulation pulses, frequency or repetition rate of stimulation pulses, pulse width of stimulation pulses, and more complex parameters such as burst definition, as are known in the art.

The CP 30 may be, for example and without limitation, a computer such as a laptop or tablet computer. The CP 30 therefore includes a microcontroller and/or microprocessor, and associated memory. The memory may take any suitable form (RAM, ROM, Flash, etc.), and stores machine readable instructions allowing the processor to perform the methods disclosed herein. To the extent Bluetooth is used as a communications protocol, the RF circuitry may be included in the device as a communications circuitry, located internal to the CP 30. If some other communications technology (inductive or Medradio) is used, or if range is limited by the IPG for example, the communications circuit may be provided via a wand having specialized circuitry (for Bluetooth, Medradio, or inductive telemetry) therein that couples, for example, to a USB port on the CP 30. The CP 30 may include a user interface, such as a screen or touchscreen, keyboard, mouse, trackball, etc. allowing the user to provide instructions and make choices.

A patient remote control (RC) 32 can be used by the patient to perform various actions relative to the IPG 10. These may be physician defined options, and may include, for example, turning therapy on and/or off, entering requested information (such as answering questions about activities, therapy benefits and side effects), and making (limited) adjustments to therapy such as selecting from available therapy programs and adjusting, for example, amplitude settings. The RC 32 can communicate via similar telemetry as the CP 30 to control and/or obtain data from the IPG 10. The patient RC 32 may also be programmable on its own, or may communicate or be linked with the CP 30. The RC 32 may be a dedicated device, including a custom device, a locked off-the-shelf device with specialized software to prevent other uses, or may be a multi-purpose device such as the patient's smart phone.

A charger 36 may be provided to the patient to allow the patient to recharge the IPG 10, if the IPG 10 is rechargeable. In some systems, the IPG 10 is not rechargeable, and so the charger 36 may be omitted. The charger 36 can operate, for example, by generating a varying magnetic field (such as via an inductor) to activate an inductor associated with the IPG 10 to provide power to recharge the IPG battery, using known methods and circuitry.

Some systems may include an external test stimulator (ETS) 38. The ETS 38 can be used to test therapy programs after the lead 12 has been implanted in the patient to determine whether therapy will or can work for the patient 16. For example, an initial implantation of the lead 12 can take place using, for example, a stereotactic guidance system, with the IPG 10 temporarily left out. After a period of healing, the patient may return to the clinic for therapy configuration and testing. The lead 12 may have a proximal end thereof connected to an intermediate connector (such as an operating room cable) that couples to the ETS 38, and the ETS 38 can be programmed using the CP 30 with various therapy programs and stimulation parameters. Once therapy suitability for the patient is established to the satisfaction of the patient 16 and/or physician, the permanent IPG 10 is implanted and the lead 12 is connected thereto, with the ETS 38 then removed from use.

A vagal stimulation system may be provided as shown at 40, located near the vagus nerve. This may be in place of the DBS IPG 10 and lead 12, if desired, or may be an additional stimulator for the patient 16. Stimulation devices may be microstimulators, and may include or exclude a lead, as desired. Some example microstimulators are can be observed in U.S. Pat. No. 8,127,424, the disclosure of which is incorporated herein by reference. Devices, including microstimulators, may be externally powered or internally powered, as desired.

FIG. 2 shows an illustrative IPG in block form. The IPG may have a suitable hermetic housing 50, which may be conductive (titanium, stainless steel, etc.) in order to serve as an additional electrode in the system. Inside housing 50 there is a power supply 52, which may include one or more batteries (rechargeable or not), along with charging circuitry (if rechargeable) and controlled voltage supplies (as desired and suitable to the system). Stimulation circuitry is shown at 54, and provides outputs for the system to use in therapy. A microcontroller 56 is also provided and may be associated with a memory 58. The microcontroller may also be in the form of a microprocessor. Any suitable arrangement of additional systems and circuitry may be included, such as additional logic, communications bus, application specific integrated circuits, etc. The memory 58 may include any of RAM, ROM, and/or Flash memory, or other memory devices/media, and stores machine readable instructions for performing the methods disclosed herein and providing device configurations as described herein. The stimulation circuitry 54 issues therapy pulses, which are directed in accordance with input/output circuitry 60 (which may include a plurality of switches to allow selection of electrodes for use in outputs), that directs signals to and receives signals from a connector block in the header 62. The connector block in the header 62 is part of port for receiving a lead as shown in FIG. 1 (above) and/or FIG. 3 (below), with individual electrical connectors for each of a plurality of electrodes on the lead(s). Typically, one to four ports are provided, for use with up to four leads, though the present innovation is not limited to a particular lead arrangement.

A communications circuit is also shown at 64. The communications circuit 64 typically includes a resonator, modulator, amplifier and antenna, and may come as a discrete chip. Commercially available chips for Medradio and/or Bluetooth (including Bluetooth Low Energy) can be used, for example. The antenna may be located in the header 62, if desired, to limit signal attenuation due to the housing 50. The communications circuit 64 provides an interface for the device to communicate with external devices including the CP and RC, which may have corresponding circuitry for using the selected communications mode.

The standard approaches to therapy in neuromodulation systems use either current controlled or voltage-controlled therapy generated by a stimulation circuitry 54. The therapy may include, for example, biphasic square waves or monophasic square waves having passive recovery. In general, the amount of current out of an electrode should zero out over time to avoid corrosion at the electrode-tissue interface. For this reason, biphasic pulses, or monophasic pulses with a passive recovery period are typically used. One or several voltage sources may be used, such as with programmable amplifiers or digital to analog conversion circuits that can convert a received therapy command into an analog output voltage, to provide voltage-controlled therapy.

Multiple independent current control (MICC) may be used as stimulation circuitry 54. MICC is a stimulus control system that provides a plurality of independently generated output currents that may each have an independent quantity of current. The use of MICC can allow spatially selective fields to be created by therapy outputs. The term “fractionalization” may refer to how the total current issued by the pulse generator via the electrodes is divided up amongst the electrodes of the lead and/or including the pulse generator canister, which can serve as an additional electrode.

For example, the device may be used with a lead as shown at 66, having a lead body carrying a plurality of electrodes at a distal end portion 68. The electrodes can be separately addressed by the stimulation circuitry 54. Eight electrodes (E1, E2 . . . . E8) are shown; more or fewer electrodes can be used, and more than one lead may be provided. A linear lead is shown at 66, other examples may use a paddle carrying two or more columns of electrodes. In other examples, and in particular for systems used as in FIG. 1 for DBS, directional electrodes can be used in which two or more electrodes, each separately addressable, are disposed about the cylindrical lead body. For example, a lead used in DBS may include a combination of segmented and ring electrodes, if desired, such as disclosed in U.S. Pat. Nos. 8,483,237 and 8,321,025, the disclosures of which are incorporated herein by reference. Fractionalization refers to the fraction of total current delivered via each electrode on the lead and/or using the housing 50, which may be a conductive electrode as well. Use of fractionalization to move a central point of stimulation to a targeted location, and/or to vary the central point of stimulation, for example, to adjust therapy or to test therapy settings, can also be referred to as current steering. Thus, for example, current outputs can be “steered” to adjust the central point of stimulation.

Some examples of current or prior versions of IPG circuitry, including in particular the stimulation circuitry 54 but also power 52, I/O 60, and microcontroller 56, as well as planned future examples, may be found in U.S. Pat. No. 10,716,932, the disclosure of which is incorporated herein by reference. Pulse generator circuitry may include that of the various commercially known implantable pulse generators for spinal cord stimulation, Vagus nerve stimulation, and deep brain stimulation as are also well known. Additional examples of circuitry, designs and operation of system devices (IPG, CP, RC, Charger, and ETS, for example) can be found, for example and without limitation, in U.S. Pat. Nos. 6,895,280, 6,181,969, 6,516,227, 6,609,029, 6,609,032, 6,741,892, 7,949,395, 7,244,150, 7,672,734, 7,761,165, 7,974,706, 8,175,710, 8,224,450, and 8,364,278, the disclosures of which are incorporated herein by reference in their entireties.

The circuitry blocks shown in FIG. 2 may be referred to as operational circuitry, and may be described using additional terms specific to particular circuitry functions thereof.

FIG. 3 shows an illustrative spinal cord stimulation SCS system as implanted. In this example, an IPG 70 may be placed near the buttocks or in the abdomen of the patient, with or without a lead extension 72 for coupling to the lead(s) 74 that enter the spinal column. Region 76 at about the level of the lower thoracic or upper lumbar vertebrae may serve as an entry point to the spinal column, where the distal end of the lead 74 with an electrode array may be placed close to the spinal cord 80. Other locations for the IPG 70 and/or lead 74 may be used. For example, sacral nerve stimulation may be performed by positioning the IPG in the lower torso, and extending a lead to near the sacral nerve, as is known in the art, or in the alternative, using a microstimulator. Peripheral nerve stimulation may also be performed, using an IPG and lead positioned at a desired location near the target neural structure, and/or using a microstimulator positioned near the target neural structure. The SCS implementation may include each of the external devices (CP, RC, Charger, ETS) identified in FIG. 1, though not shown in FIG. 2.

FIGS. 4A-4B illustrate waveforms that are known for use in neuromodulation. The Figures are illustrative and not intended to be limiting. FIG. 4A shows the delivery of charge balanced, biphasic square waves, which are commonly used. A biphasic waveform has a positive phase 100 and a negative phase 102, delivered in relatively quick succession (some minimal off time may separate the two). The waveform has an amplitude 104, and a pulse width 106, each of which are equal to one another.

The leading edge of each pair is separated by a period 108. For tonic stimulation, the period 108 is constant. When describing a neuromodulation therapy, the frequency, which is the inverse of the period 108, may be used. Systems known in the art may deliver therapy at frequencies in the range of 0 to 1200 Hz, or even up to 10,000 Hz, or higher. Frequencies used in different therapy locations (VNS, DBS, SCS, etc.) may vary. For example, DBS is commonly delivered in the range of up to a few hundred Hz, while some SCS systems use 10 kHz. The present invention is not limited to a particular frequency range, and this discussion is provided merely for context.

More complex patterns can be used, including varying amplitude, pulse width, and/or period 108 from one pair to the next. Charge balancing is commonly used to prevent muscle stimulation and/or electrode-tissue interface breakdown (corrosion) due to charge buildup. Waveform patterns may be developed in which the period 108 changes from one pair to the next, as well as other parameters including pulse width 106 and amplitude 104. For example, at the start of therapy delivery, it is known to ramp the amplitude upward over the course of several pulse pairs from a low value up to the intended therapy amplitude. Ramped, triangular, sinusoidal, monophasic and other stimulation types may be used as desired.

The waveform of FIG. 4A may be characterized as having an active recharge, in which a pulse is intentionally delivered at 102 with reverse polarity relative to pulse 100. Passive recharge can be used as shown in FIG. 4B. At the end of the first phase 110, a passive recharge 112 begins in which the output electrode pair is grounded, causing charge accumulated during the first phase flow back into the circuit. If this was to be observed as a current flow, one would observe the sloping reverse flow shown in FIG. 4B. As noted, other waveforms may be used. Some examples may instead use a continuous waveform, such as a sinusoidal or other shaped waveform.

Some systems are current controlled, meaning that the output circuitry is configured to issue pulses at a set current amplitude, regardless of the impedance encountered by the pulse (within physical bounds). A voltage-controlled system instead controls the output voltage, regardless of impedance and current output (again, within physical bounds). The present disclosure is not limited to either waveform type nor by the other parameters noted above.

Each of these non-limiting examples can stand on its own, or can be combined in various permutations or combinations with one or more of the other examples. The above detailed description includes references to the accompanying drawings, which form a part of the detailed description. The drawings show, by way of illustration, specific embodiments and/or “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” Moreover, in the claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, innovative subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the protection should be determined with reference to the appended claims, along with the full scope of equivalents to which such claims are entitled.

What is claimed is:

1. A method of delivering a neuromodulation therapy comprising:
applying a stimulation pattern to the patient using an implantable pulse generator attached to an implantable lead, the lead having a distal end carrying a plurality of electrodes, the distal end positioned near a neural tissue to be stimulated;
modifying the applied stimulation pattern using a separately defined adjustment parameter for adjusting the applied stimulation pattern;
wherein the adjustment parameter changes in response to at least one of:
a daily temporal pattern; or
a patient event.

2. The method of claim 1, wherein the separately defined adjustment parameter defines an amplitude as a function of time, and the step of modifying the applied stimulation pattern is performed by adjusting an amplitude of the applied stimulation pattern as a function of time.

3. The method of claim 1, wherein the adjustment parameter changes in response to each of a daily temporal pattern and a patient event by:
defining at least first and second time periods, separated by a patient event;
prior to the patient event, controlling amplitude of the applied stimulation in response to passage of time according to a first shape;
determining the patient event has occurred; and
after the patient event, controlling amplitude of the applied stimulation in response to passage of time according to a second shape.

4. The method of claim 3, wherein the patient event is a medication being received or consumed by the patient.

5. The method of claim 3, wherein determining the patient event has occurred comprises receiving a communication from a patient remote control configured to be used by the patient to communicate with the implantable pulse generator.

6. The method of claim 3, wherein the patient event is onset of sleep or end of sleep.

7. The method of claim 1, wherein the implantable pulse generator comprises operational circuitry configured to perform the method, such that the method occurs without communication from a clinician programmer adapted to communicate with the implantable pulse generator.

8. The method of claim 1, further comprising:
receiving a patient modification from the patient for modifying the applied stimulation pattern;
modifying the applied stimulation pattern using the patient modification to yield a modified and adjusted stimulation parameter; and
determining whether to retain the patient modification based on comparison of the patient modification to one or more boundary conditions.

9. The method of claim 8, wherein the step of determining whether to retain the patient modification is performed as follows:
the boundary conditions include each of a first boundary and a second boundary;
if the modified and adjusted stimulation parameter does not exceed the first boundary, the patient modification is retained and used to modify the applied stimulation pattern until a further patient modification is received, or the implantable pulse generator is reprogrammed;
if the modified and adjusted stimulation parameter exceeds the second boundary, the patient modification is used only until one of the following occurs:
an inflection point in the daily temporal pattern is reached, and
another patient event occurs.

10. The method of claim 1, wherein the separately defined adjustment parameter is used to modify at least one of amplitude, pulse width, or frequency of the applied stimulation pattern.

11. An implantable medical device system comprising an implantable medical device (IMD) configured to generate therapy outputs, and a patient remote control (RC) configured to communicate with the IMD, the system configured to operate the following method:
receiving at the RC a request from the patient to modify a therapy output parameter;
analyzing the request and either:
determining the request is within a first set of parameter limits and, if so, modifying a programmed setting for the therapy parameter so that the IMD will continue to use the modified programmed setting for the therapy parameter until changed or reset; or
determining the request is outside the first set of parameter limits and, if so, modifying the therapy output temporarily and then returning to the programmed setting.

12. The system of claim 11, further configured to modify the therapy output temporarily by modifying the therapy output for a fixed period of time, and then returning to the programmed setting.

13. The system of claim 11, wherein the programmed setting varies over time, and the modified programmed setting is mathematically generated to modify a plurality of values of the programmed setting.

14. The system of claim 11, wherein the RC is configured to perform the analyzing and determining steps, and issues a communication to the IMD to cause the IMD to modify the programmed setting or to modify the therapy output temporarily.

15. The system of claim 11, wherein the RC is configured to communicate the request from the patient to the IMD, and the IMD is configured to analyze the request and perform the determining steps.

16. A method of testing a therapy program on a patient, comprising:
defining the therapy program, including within the therapy program one or more amplitude maximums and one or more amplitude or pattern changes, separated by one or more periods of stimulation;
identifying plateau periods as intervals in the therapy program during which none of the one or more amplitude maximums occur and none of the identified amplitude or pattern changes occur, the plateau periods having durations and plateau therapy parameters;
creating compressed periods by reducing the reducing the plateau period durations without modifying the plateau therapy parameters; and
applying a test program to the patient, the test program being the same as the therapy program except that the plateau periods are replaced by the compressed periods.

17. The method of claim 16, wherein the compressed periods are reduced in duration by at least 75% relative to the plateau periods.

18. The method of claim 16, wherein the step of identifying plateau periods comprises:
identifying a predetermined number of amplitude maximums, and defining extreme periods for the predetermined number of amplitude maximums, the extreme periods having extreme period durations and extreme period therapy parameters;
defining transition periods for the predetermined number of transition period in which the one or more amplitude or pattern changes occur, the transition periods having transition period durations and transition period therapy parameters.

19. The method of claim 16, further comprising analyzing the transition periods against a slope threshold, and, if the slope threshold exceeds a slope of a first transition period, compressing the first transition period.

20. The method of claim 16, wherein the therapy program is a neuromodulation therapy program, and the step of applying the test program comprises either delivering neuromodulation by deep brain stimulation, or by spinal cord stimulation.
